(19) United States
(12) Patent Application Publication
(10) Pub. No.: US 2004/0219204 A1
(43) Pub. Date: Nov. 4, 2004

(54) Method of treating angiogenic tissue growth

(76) Inventors: Ken Shi Kun Huang, Castro Valley, CA (US); Anthony Huang, Saratoga, CA (US); Francis J. Martin, San Francisco, CA (US)

Correspondence Address: ALZA Corporation, Patent Law Department, M10-3, 1900 Charleston Road, Mountain View, CA, 94049, US

(21) Appl. No.: 10/742,074

(22) Filed: Dec. 19, 2003

Related U.S. Application Data
(60) Provisional application No. 60/435,115, filed on Dec. 19, 2002.
(60) Provisional application No. 60/488,143, filed on Jul. 16, 2003.

(51) Int. Cl. A61K 9/127; A61K 33/24; A61K 38/18

(52) U.S. Cl. 424/450; 424/649; 514/12

(57) ABSTRACT

A method of treating a disease or disorder characterized by angiogenic tissue growth is described. The method includes providing an immunoliposome composition comprised of (i) vesicle-forming lipids including between 1-20 mole percent of a vesicle-forming lipid derivatized with a hydrophilic polymer, (ii) a targeting ligand having biological activity to promote angiogenesis, and (iii) a drug entrapped in said liposomes; and administering the immunoliposome composition to a subject.

This application claims the benefit of U.S. Provisional Application No. 60/435,115, filed Dec. 19, 2002 and of U.S. Provisional Application No. 60/488,143, filed Jul. 16 2003, both of which are incorporated herein by reference in their entirety.

### FIELD OF THE INVENTION

The present invention relates to a method of arresting angiogenic tissue growth by administering liposomes that include a targeting ligand having biological activity to stimulate angiogenesis.

### BACKGROUND OF THE INVENTION

Angiogenesis, the development of a new blood supply, is an essential process in the development, growth and metastasis of human tumors. The process of angiogenesis consists of a series of interactive events: quiescent endothelial cells in normal blood vessels (adjacent to a nascent tumor or micrometastatic lesion) are stimulated by angiogenic factors to degrade the underlying basement membrane, to migrate within the interstitial matrix, to proliferate and to organize themselves into tubular structures which become mature blood vessels (Gasparini, G., Drugs, 58(1):17 (1999)). As these new vessels sprout, and before they seal into closed tubes, defects or gaps appear in the endothelium. The increased understanding of angiogenesis has led to the tumor vasculature becoming an attractive target for anti-cancer intervention strategies. These strategies have focuses on inhibiting the proliferation of tumor angiogenic endothelial cells and thereby interrupting the flow of essential nutrients to tumor cells (anti-angiogenesis therapy).

Proliferating vascular endothelial cells up-regulate the expression of growth factor receptors, including basic fibroblast growth factor receptor (bFGFr), epithelial cell growth factor receptor (FGFr), and vascular endothelial cell growth factor receptor (VEGFr) (Chandler, L. A. et al., Int. J. Cancer, 81(3):451 (1999). The high-affinity binding of the cognate ligand to one of these receptors is followed by internalization of the ligand-receptor complex and stimulation of intracellular pathways leading to cell proliferation. Such paracrine/autocrine proliferation of the vascular endothelial cells is the hallmark of the angiogenesis process.

These growth factors initiate growth of new cancers and also stimulate existing cancers and hyperproliferative disorders. There is a direct correlation between the circulating level of certain growth factors and cancer proliferation. Prior art approaches to cancer therapy have attempted to inhibit the growth-promoting activity of growth factors, for example by providing antibodies specific for growth factors to reduce of inhibit their activity (U.S. Pat. No. 5,919,459).

Liposomes have proved to be a valuable therapeutic tool for delivery of drugs, particularly for cancer chemotherapeutic agents. In particular, liposomes with an external coating of hydrophilic polymer chains are able to circulate for several days after intravenous administration, and are small enough to pass out of blood vessels (extravasate) which have compromised endothelial barriers. It has been recently shown that such long-circulating liposomes are able to extravasate into newly sprouting vessels formed during tumor angiogenesis, as these vessels are often incompletely formed into closed tubes, or have gaps and other defects in the endothelium (Yuan, F., et al., Cancer Res., 54(13):3352 (1994)).

Liposomes loaded with cytotoxic drugs can be “sensitized” or covalently conjugated with ligands on their outer surface to target the liposomes to a particular cell, tissue, or receptor in the body (see, for example, U.S. Pat. Nos. 6,214,388; 6,316,024; 6,056,973; 6,043,094). Liposomes bearing such ligands on their surface, referred to as immunoliposomes, bind specifically and with high affinity to the surfaces of cells expressing the complimentary receptor. Approaches to preparing immunoliposomes are described in the art, and include attaching the ligand to the polar head group of the lipid forming the liposomes (U.S. Pat. No. 5,013,556; Klibanov, A. L., et al., Biochim. Biophys. Acta., 1062:142-148 (1991); Hansen, C. B., et al., Biochim. Biophys. Acta, 1239:133-144 (1995)) or attaching the ligand to the distal end of all or a portion of the hydrophilic polymer chains surrounding the liposome (Allen. T. M., et al., Biochim. Biophys. Acta, 1237:99-108 (1995); Blume, G. , et al., Biochim. Biophys. Acta, 1149:180-184 (1993)). Attachment of the targeting ligand to the distal end of the polymer chains can be achieved via several methods, including preparation of lipid vesicles which include an end-functionalized lipid-polymer derivative; that is, a lipid-polymer conjugate where the free polymer end is reactive or “activated”. Such an activated conjugate is included in the liposome composition and the activated polymer ends are reacted with a targeting ligand after liposome formation. Alternatively, the lipid-polymer-ligand conjugate can be included in the lipid composition at the time of liposome formation. A third method involves incubation of pre-formed liposomes with a micellar solution of lipid-polymer-ligand conjugates to achieve insertion of the conjugate into the liposomes (U.S. Pat. No. 6,316,024; 6,214,388).

While much progress has been made in the field of immunoliposomes, there remains a need for new strategies for targeting liposomes to a specific tissue for therapy. There remain a particular need for a strategy for tumor therapy.

### SUMMARY OF THE INVENTION

In one aspect, the invention includes a method of treating a disease or disorder characterized by angiogenic tissue growth, comprising providing an immunoliposome composition comprised of (i) vesicle-forming lipids including between 1-20 mole percent of a vesicle-forming lipid derivatized with a hydrophilic polymer, (ii) a targeting ligand having biological activity to promote angiogenesis, and (iii) an drug entrapped in said liposomes; and administering the immunoliposome composition to a subject suffering from such a disease or disorder.

In one embodiment, the targeting ligand is a growth factor. Exemplary growth factors include fibroblast growth factor (FGF), epidermal growth factor (EGF), and vascular endothelial cell growth factor (VEGF).

In another embodiment, the drug entrapped in the immunoliposomes is a cytotoxic chemotherapeutic agent. Exemplary agents include platinum coordination complexes, such as cisplatin, anthracycline antibiotics, and vinca alkaloids. In another embodiment, the drug entrapped in the immunoliposomes is an angiogenic inhibitor. Exemplary antiangiogenic agents include FGFR kinase inhibitors, EGFR kinase inhibitors, VEGFR kinase inhibitors, matrix metalloproteinase inhibitors. Specific exemplary antiangiogenesis agents include marmiastat, prinomastat, BMS275291, BAY12-9566, neovastat, rhuMAb VEGF, SU5416, SU6668, ZD6474, CP-547, CP-632, ZD4190, endostatin, thalidomide and thalidomide analoges, sqalamine, celecoxib, ZD6126, and TNP-470.

In yet another embodiment, the hydrophilic polymer is polyethylene glycol.

In one embodiment, the immunoliposome includes as the polymer polyethylene glycol; the ligand is basic fibroblast growth factor; and the drug is a chemotherapeutic agent.

In another embodiment, the immunoliposome includes as the polymer polyethylene glycol; the ligand is basic fibroblast growth factor; and the drug is an angiogenic inhibitor.

These and other objects and features of the invention will be more fully appreciated when the following detailed description of the invention is read in conjunction with the accompanying drawings.

### BRIEF DESCRIPTION OF THE DRAWINGS

FIGS. 1A-1E are computer-generated photomicrographs of cells showing binding and internalization after incubation for 30 minutes at 4° C. or for 1 hour and 3 hours at 37° C. of liposomes (FIGS. 1A-1C) and FGF-immunoliposomes (FIGS. 1D-1E);

FIGS. 2A-2B are graphs showing inhibition of cell growth (expressed as percentage of untreated, control cells) as a function of drug concentration for cells treated with drug entrapped in PEG-coated liposomes (circles), drug entrapped in FGF-immunoliposomes (squares), placebo FGF-immunoliposomes (triangles), or with micelles of lipid-PEG-FGF (diamonds), where the drug was doxorubicin (FIG. 2A) or cisplatin (FIG. 2B);

FIGS. 3A-3B are computer-generated photomicrographs of tissue sections after staining for Lewis lung tumor vasculature angiogenic endothelial cells, where the angiogenic cells lining the tumor vasculature were identified by anti-CD34 receptor antibody binding (FIG. 3A) and the cells expressing FGF receptors were identified by anti-FGF receptor antibodies (FIG. 3B);

FIG. 4 is a graph of tumor size, in mm³, as a function of days after implantation of Lewis lung tumor xenografts in mice treated with saline (diamonds), PEG-coated liposomes with entrapped doxorubicin (open squares), PEG-coated liposomes with entrapped cisplatin (solid squares), FGF-immunoliposomes with entrapped doxorubicin (open circles), or FGF-immunoliposomes with entrapped cisplatin (solid circles).

### DETAILED DESCRIPTION OF THE INVENTION

### I. Definitions

The terms “vesicle-forming lipid” and “amphipathic vesicle-forming lipid” intend (a) any amphipathic lipid having hydrophobic and polar head group moieties, and which by itself can form spontaneously into bilayer vesicles in water, as exemplified by phospholipids, or (b) is stably incorporated into lipid bilayers in combination with phospholipids with its hydrophobic moiety in contact with the interior, hydrophobic region of the bilayer membrane, and any polar region oriented toward the exterior, polar surface of the membrane. An example of the latter type of vesicle-forming lipid is cholesterol and cholesterol derivatives such as cholesterol sulfate and cholesterol hemisuccinate.

The terms “ligand” or “targeting moiety”, as used herein, refer generally to all molecules capable of specifically binding to a particular target molecule and forming a bound complex. The ligand and its corresponding target molecule form a specific binding pair.

The term “immunoliposome” refers to a liposome bearing a moiety that acts as a targeting ligand enabling the liposome to specifically bind to a particular “target” molecule that may exist in solution or may be bound to the surface of a cell.

A “hydrophilic polymer” as used herein refers to long chain highly hydrated flexible neutral polymers. Polyethylene glycol is the classic example of a hydrophilic polymer, however many others are described in the art, for example in U.S. Pat. No. 5,395,619 and U.S. Pat. No. 5,631,018, which are incorporated by reference herein.

The term “mole percent” when referring to the percentage of hydrophilic polymer in a liposome is expressed relative to the total lipid in the liposome unless otherwise stated. Thus, for example, in a liposome comprising a ratio of phosphatidylcholine (PC) to cholesterol (Chol) of 150:100, a 4 mole percent of hydrophilic polymer (e.g. PEG) would represent a ratio of PC:Chol:PEG of about 150:100:10.

### II. Method of Treatment

In one aspect, the invention provides for a method of treating a disorder or disease characterized by angiogenic tissue growth. As discussed above, angiogenesis involves the process of new blood vessel development and formation and plays an important role in numerous physiological events, both normal and pathological. The naturally occurring balance between endogenous stimulators and inhibitors of angiogenesis is one in which inhibitory influences predominate (Rastinejad et al., Cell, 56:345 (1989)). Neovascularization is part of a normal physiological condition in certain situations, such as wound healing, organ regeneration, embryonic development, and female reproductive processes, and angiogenesis is stringently regulated and spatially and temporally delimited. However, under conditions of pathological angiogenesis these regulatory controls fail. Such unregulated angiogenesis becomes pathologic and sustains progression of many neoplastic and non-neoplastic diseases. A number of serious diseases are dominated by abnormal neovascularization including solid tumor growth and metastases, arthritis, some types of eye disorders, and psoriasis. See, e.g., reviews by Moses et al., Biotech., 2:630 (1991); Folkman et al., N. Engl. J. Med., 333:1757 (1995); Auerbach et al., J. Microvasc. Res., 29:401 (1985); Folkman, ADVANCES IN CANCER RESEARCH, eds. Klein and Weinhouse, Academic Press, New York, pp. 175-203 (1985); Patz, Am. J. Opthalmol. 94:715 (1982); Folkman et al., Science, 221:719 (1983).

In a number of pathological conditions, the process of angiogenesis contributes to the disease state. For example, significant data have accumulated which suggest that the growth of solid tumors is dependent on angiogenesis (Folkman and Klagsbrun, Science, 235:442 (1987)). The maintenance of the avascularity of the cornea, lens, and trabecular meshwork is crucial for vision as well as to ocular physiology. There are several eye diseases, many of which lead to blindness, in which ocular neovascularization occurs in response to the diseased state. These ocular disorders include diabetic retinopathy, neovascular glaucoma, inflammatory diseases and ocular tumors (e.g., retinoblastoma). There are also a number of other eye diseases which are also associated with neovascularization, including retrolental fibroplasia, uveitis, retinopathy of prematurity, macular degeneration, and approximately twenty eye diseases which are associated with choroidal neovascularization and approximately forty eye diseases associated with iris neovascularization. See, e.g., reviews by Waltman et al., Am. J. Ophthal., 85:704 (1978) and Gartner et al., Surv. Ophthal., 22:291 (1978).

Accordingly, the present invention provides a method for treating conditions such as those describe above, and others, that are associated with angiogenic neovascularization. The method involves providing a targeted, long-circulating liposome composition having cytotoxic activity against the proliferating cells. Targeting of the composition is achieved by a ligand that is active in its native form, i.e., when unconjugated to a liposome, in the cascade of events associated with angiogenesis. Selection of a ligand having biological, stimulatory activity for angiogenic neovascularization is opposite of the intended therapeutic activity of arresting continued growth of cells associated with the disease or disorder. The invention contemplates selecting a targeting ligand having angiogenic activity for targeting to the angiogenic tissue a drug-laden liposome having a cytotoxic effect. Rather than targeting the liposomes directly to the disease or disorder, the liposomes are targeted to tissue associated with the disease or disorder. In this way, the liposomes provide an indirect method of treating a disease or disorder.

Targeting ligands having growth-stimulatory activity in angiogenesis include growth factors, in particular fibroblast growth factors. The fibroblast growth factor (FGF) family consists of at least eighteen distinct members (Basilico et al., Adv. Cancer Res., 59:115 (1992); Fernig et al., Prog. Growth Factor Res., 5(4):353 (1994)) which generally act as mitogens for a broad spectrum of cell types. For example, basic FGF (also known as FGF-2) is mitogenic in vitro for endothelial cells, vascular smooth muscle cells, fibroblasts, and generally for cells of mesoderm or neuroectoderm origin, including cardiac and skeletal myocytes (Gospodarowicz et al., J. Cell. Biol., 70:395 (1976); Gospodarowicz et al., J. Cell. Biol., 89:568 (1981); Kardami, J. Mol. Cell. Biochem., 92:124 (1990)). In vivo, bFGF has been shown to play a role in avian cardiac development (Sugi et al., Dev. Biol., 168:567 (1995); Mima et al., Proc. Nat'l. Acad. Sci., 92:467-471 (1995)), and to induce coronary collateral development in dogs (Lazarous et al., Circulation, 94:1074 (1996)). In addition, non-mitogenic activities have been demonstrated for various members of the FGF family. Non-proliferative activities associated with acidic and/or basic FGF include: increased endothelial release of tissue plasminogen activator, stimulation of extracellular matrix synthesis, chemotaxis for endothelial cells, induced expression of fetal contractile genes in cardiomyocytes (Parker et al., J. Clin. Invest., 85:507 (1990)), and enhanced pituitary hormonal responsiveness (Baird et al., J. Cellular Physiol., 5:101 (1987)).

Ligands from the FGF family that are contemplated for use in the method described herein include Fibroblast Growth Factor (FGF), EGF (Epidermal Growth Factor), Vascular Endothelial Cell Growth Factor (VEGF), and fragments of these proteins that retain binding and biological activity. A preferred ligand is FGF.

The ligand selected for use preferably also is one that is internalized by the cell after binding with the cellular receptor. Binding of the liposome-attached ligand may have growth-stimulating activity; however, uptake of the ligand and attached liposome into the cell delivers the cytotoxic agent to the cell for intracellular activity. As will be illustrated below, drug-laden immunoliposomes targeted using a growth-stimulatory ligand are readily internalized by the cells and result in an enhanced suppression of cell growth.

1. Liposome Composition

The immunoliposomes are comprised primarily of vesicle-forming lipids. Vesicle-forming lipids can form spontaneously into bilayer vesicles in water, as exemplified by the phospholipids. The liposomes can also include other lipids incorporated into the lipid bilayers, with the hydrophobic moiety in contact with the interior, hydrophobic region of the bilayer membrane, and the head group moiety oriented toward the exterior, polar surface of the bilayer membrane.

The vesicle-forming lipids are preferably ones having two hydrocarbon chains, typically acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids include glycolipids and sterols such as cholesterol.

The liposomes also include a vesicle-forming lipid derivatized with a hydrophilic polymer. As has been described, for example in U.S. Pat. No. 5,013,556 and in WO 98/07409, which are hereby incorporated by reference, such a hydrophilic polymer provides a surface coating of hydrophilic polymer chains on both the inner and outer surfaces of the liposome lipid bilayer membranes. The outermost surface coating of hydrophilic polymer chains is effective to provide a liposome with a long blood circulation lifetime in vivo. The inner coating of hydrophilic polymer chains extends into the aqueous compartments in the liposomes, i.e., between the lipid bilayers and into the central core compartment, and is in contact with the entrapped compound.

Vesicle-forming lipids suitable for derivatization with a hydrophilic polymer include any of those lipids listed above, and, in particular phospholipids, such as distearoyl phosphatidylethanolamine (DSPE). Hydrophilic polymers suitable for derivatization with a vesicle-forming lipid are well known in the art and described in U.S. Pat. No. 5,395,619 and U.S. Pat. No. 5,631,018. A preferred hydrophilic polymer chain is polyethyleneglycol (PEG), preferably as a PEG chain having a molecular weight between 500-10,000 Daltons, more preferably between 500-5,000 Daltons, most preferably between 1,000-2,000 Daltons. Methoxy or ethoxy-capped analogues of PEG are also preferred hydrophilic polymers, commercially available in a variety of polymer sizes, e.g., 120-20,000 Daltons. Preparation of vesicle-forming lipids derivatized with hydrophilic polymers has been described, for example in U.S. Pat. No. 5,395,619. Preparation of liposomes including such derivatized lipids has also been described (U.S. Pat. No. 5,013,556), where typically, between 1-20 mole percent of such a derivatized lipid is included in the liposome formulation.

The liposomes may be prepared by a variety of techniques, such as those detailed in Szoka, F., Jr., et al., Ann. Rev. Biophys. Bioeng. 9:467 (1980). Typically, the liposomes are multilamellar vesicles (MLVs), which can be formed by simple lipid-film hydration techniques. In this procedure, a mixture of liposome-forming lipids and including a vesicle-forming lipid derivatized with a hydrophilic polymer are dissolved in a suitable organic solvent which is evaporated in a vessel to form a dried thin film. The film is then covered by an aqueous medium to form MLVs, typically with sizes between about 0.1 to 10 microns. Exemplary methods of preparing derivatized lipids and of forming polymer-coated liposomes have been described in U.S. Pat. Nos. 5,013,556, 5,631,018 and 5,395,619, which are incorporated herein by reference.

The drug is incorporated into liposomes by standard methods, including (i) passive entrapment of a water-soluble compound by hydrating a lipid film with an aqueous solution of the agent, (ii) passive entrapment of a lipophilic compound by hydrating a lipid film containing the agent, and (iii) loading an ionizable drug against an inside/outside liposome ion gradient, termed remote loading. Other methods, such as reverse evaporation phase liposome preparation, are also suitable.

The drug entrapped in the immunoliposome, in one embodiment, is a cytotoxic agent, including but not limited to vinca alkaloids (e.g., vinblastine, vincristine), anthracycline antibiotics (e.g., doxorubicin, daunorubicin, epirubicin) antibiotics (e.g., bleomycin, mitomycin, dactinomycin), platinum compounds (cisplatin, carboplatin, oxaliplatin, nedaplatin, etc.), topoisomerase inhibitors (camptothecin, topotecan, GG211, CKD506, etc.), alkylating agents (nitrogen mustards such as melphalan, chlorambucin, nitrosoureas such as carmustine, lomustine), pyrimidine analogs (fluorouracil, cytarabine), and the like.

In another embodiment, the drug entrapped in the immunoliposome is an angiogenic inhibitor. Angiogenesis involves a series of complex and interrelated steps. The molecular events that sustain angiogenesis are targets for inhibitor agents. Various agents are known that target these different steps involved in angiogenesis. Drug that inhibit matrix breakdown, matrix metalloproteinase inhibitors, are one class of drugs exemplified by the inhibitors marmiastat, prinomastat, BMS275291, BAY12-9566, and neovastat (Shepherd, F. A., Lung Cancer, 41(Suppl. 1):S63 (2003)). Drugs that block endothelial cell signaling via vascular endothelial growth factor and its receptor include rhuMAb VEGF, SU5416, SU6668, ZD6474, CP-547, CP-632, and ZD4190 (Shephard, F. A, Id.). Other drugs are similar to endogenous inhibitors of angiogenesis, such as interferons, angiostatin, troponin I, and endostatin. Thalidomide and thalidomide analoges, sqalamine, celecoxib, fenretinide, ZD6126, and TNP-470 are other antiangiogenic inhibitors. Additional exemplary antiangiogenic agents include antibodies and small molecule FGFR kinase inhibitors, EGFR kinase inhibitors, and VEGFR kinase inhibitors. Bevacizumab is one such inhibitor that acts as a vascular endothelial growth factor antagonist.

Administration of liposomes and immunoliposomes is well known in the art, and depending on the condition to be treated and the nature of the preparation, liposomal compositions can be administered via injection intravenously, subcutaneously, intramuscularly, etc., or given via inhalation, for systemic or local delivery. Other routes are contemplated and known to those of skill in the art.

2. Exemplary Composition and Method of Treatment

Immunoliposomes having an FGF2 targeting ligand were prepared as described in Example 1. Liposomes having an entrapped cytotoxic agent, doxorubicin or cisplatin, were prepared by known techniques. Doxorubicin-containing liposomes were prepared from HSPC, cholesterol, and mPEG-DSPE and the drug was then remotely loaded into the liposomes against an ammonium sulfate gradient, as has been described (U.S. Pat. Nos. 5,013,556; 5,316,771). Cisplatin-containing liposomes were prepared from HSPC, cholesterol, and mPEG-DSPE, where the cisplatin was entrapped via passive encapsulation as described in U.S. Pat. No. 5,945,122. The FGF2 targeting ligand was inserted into the preformed liposomes by incubating micelles of FGF2-PEG-DSPE with the suspensions of the preformed liposomes. The immunoliposome preparations included a small amount of Texas-Red-DSPE conjugate to permit tracking and visualization of the immunoliposomes.

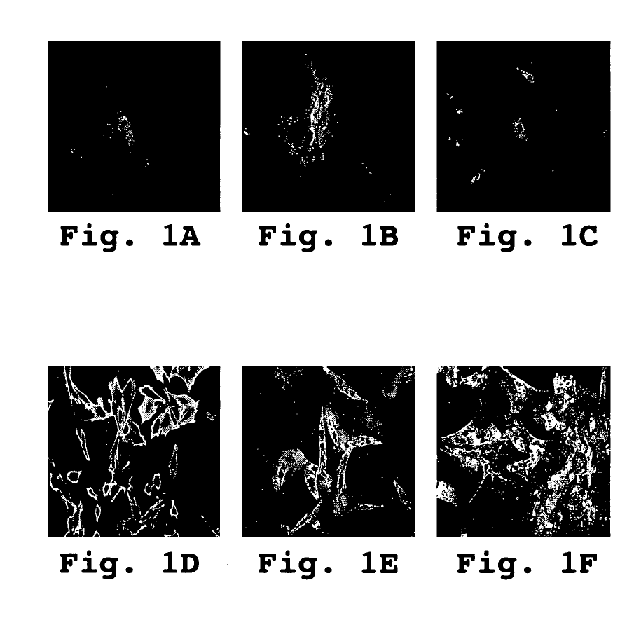

Specific binding and internalization of FGF2 immunoliposomes into baby hamster kidney (BHK) cells was evaluated by incubating the immunoliposomes with BHK cells expressing FGF2 receptor, as described in Example 1. The cells were visualized via confocal fluorescence microscopy after 30 minutes of incubation at 4° C. where the cells had no endocytic activity, or for 1 hour or 3 hours at 37° C. The results are shown in FIGS. 1A-1F, where FIGS. 1A-1C correspond to pegylated-liposomes (lacking an FGF2 targeting ligand) and FIGS. 1D-1E correspond to immunoliposomes. Very little fluorescent signal was detected on the surface or inside BHK cells after incubation with pegyalted liposomes lacking a targeting ligand (FIGS. 1A-1C). After 30 minutes of incubation at 4° C., immunoliposomes were located mainly on or near the cell surface with little observable fluorescence in the cell cytoplasm, as seen in FIG. 1 D. After incubation at 37° C. for 1 hour or 2 hours of the cells with immunoliposomes, fluorescent signal was detected throughout the cytoplasm, in particular in the endocytic vesicles, as seen in FIGS. 1E-1F.

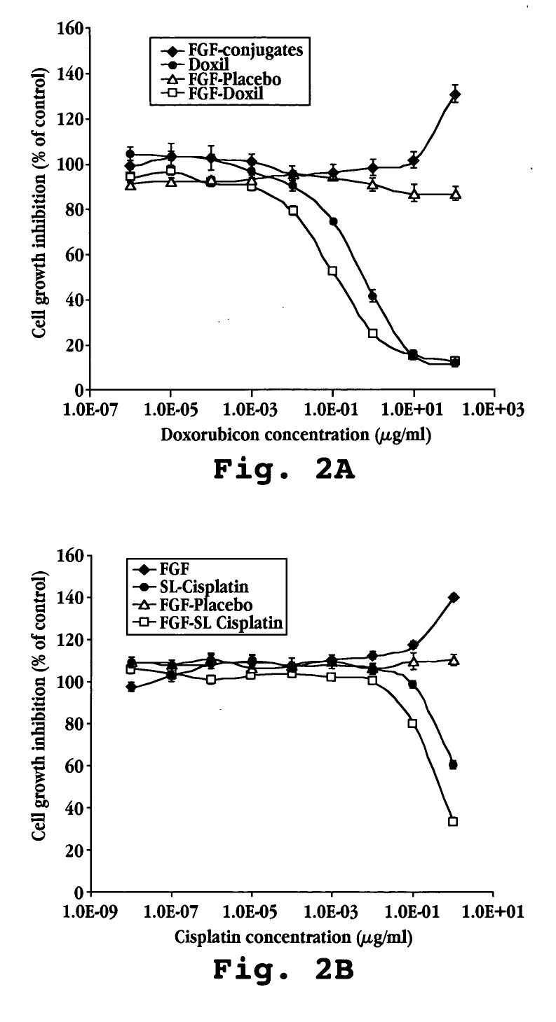

In another study, the ability of the immunoliposomes to inhibit cell growth, relative to pegylated liposomes lacking a targeting ligand, was evaluated. As described in Example 1, BHK cells were incubated with the immunoliposome compositions or with pegylated liposome compositions for 72 hours in culture, after which cell numbers were determined using MTT assay. FIGS. 2A-2B show the results, where the inhibition of cell growth (expressed as percentage of untreated, control cells) is plotted as a function of drug concentration for cells treated with drug entrapped in PEG-coated liposomes (circles), drug entrapped in FGF-immunoliposomes (squares), placebo FGF-immunoliposomes (triangles), or with micelles of lipid-PEG-FGF (diamonds). The data in FIG. 2A corresponds to the doxorubicin preparations and the data in FIG. 2B corresponds to the cisplatin preparations.

FIGS. 2A-2B show that binding and internalization of FGF2 on the immunoliposomes resulted in enhanced delivery of cytotoxic agent to the cells. Treatment of BHK cells with FGF2-targeted liposomes resulted in greater growth inhibition relative to treatment with pegylated liposomes lacking the FGF2 targeting ligand (circles), with placebo liposomes having an FGF2 targeting moiety (triangles), or with the FGF2-PEG-DSPE targeting conjugate (diamonds).

In vivo studies were conducted to evaluate the effect of immunoliposomes containing a targeting ligand that stimulates angiogenic tissue growth, FGF2, and a cytotoxic agent on tumor growth. As described in Example 2, mice were inoculated subcutaneously with Lewis lung carcinoma cells. One group of mice was selected for removal of tumor for immunohistochemical studies. The remaining mice were treated with pegylated liposomes lacking an FGF2 targeting ligand or immunoliposomes.

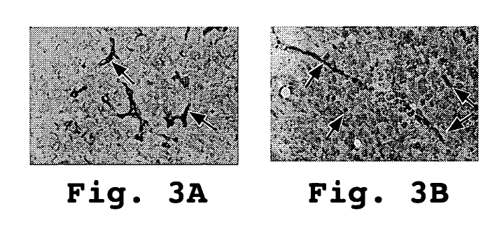

FIGS. 3A-3B are photomicrographs after immunohistochemical staining. Sections of the tumors resulting from inoculation of Lewis lung carcinoma cells were taken and the angiogenic endothelial cells and cells expressing FGF2 receptors were examined by immunohistochemical staining. FIG. 3A shows a photomicrograph of a tumor tissue section after treating with anti-CD-34 receptor antibodies to angiogenic endothelial cells. The arrows in the figure identify angiogenic endothelial cells along a blood vessel. FIG. 3B shows a photomicrograph of a tumor tissue section after treating with anti-FGF receptor antibodies. The photomicrographs show that angiogenic endothelial tissue lining the tumor vasculature were heavily labeled with anti-FGF2 receptor antibodies, while fewer of the Lewis lung tumor cells bound anti-FGF receptor antibodies.

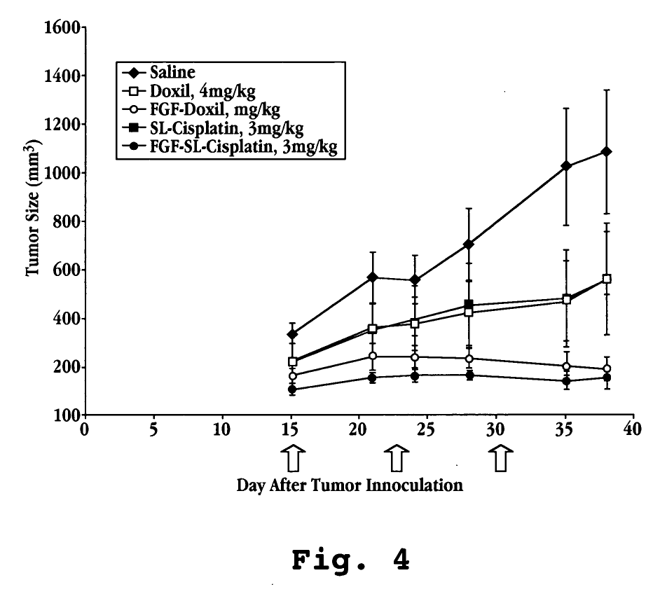

FIG. 4 shows the growth of Lewis lung tumor xenografts in mice after treatment with saline (diamonds), pegylated liposomes with entrapped doxorubicin (open squares), pegylated liposomes with entrapped cisplatin (solid squares), FGF-immunoliposomes with entrapped doxorubicin (open circles), or FGF-immunoliposomes with entrapped cisplatin (solid circles). Treatment with pegylated liposomes lacking an FGF2 targeting reduced tumor growth compared to the saline control, although the average tumor size increased 2.5 fold over the course of treatment. In contrast, average tumor sizes remained stable in the animals treated with FGF2-immunoliposomes containing doxorubicin or cisplatin. FGF2-immunoliposomes were significantly more effective in inhibiting tumor growth than the pegylated liposomes lacking an FGF2 targeting moiety.

The data in FIGS. 3A-3B and FIG. 4 taken together show that the tumor growth suppression after treatment with FGF2-immunoliposomes was due to an anti-angiogenesis effect rather than a direct cytotoxic effect on the tumor cells, since the expression of FGF-receptors on Lewis lung tumor cells was low (FIG. 3B).

From the foregoing, it can be seen how various objects and features of the invention are met. The method of the invention provides a therapy effective to prevent the development, growth, and metastasis of human neoplastic disease. Immunoliposomes having a targeting ligand that stimulates angiogenic tissue growth and having an entrapped cytotoxic drug were effective to inhibit growth of tumors having little receptor expression for the targeting ligand. The liposomal ligand mediates the binding and internalization of the immunoliposome via an interaction with receptors expressed on angiogenic vascular endothelial cells and subsequent introduction of the cytotoxic agent into the cellular cytoplasm. The immunoliposomes have activity to directly target proliferating vascular endothelial cells which are essential for tumor angiogenesis. FGF-sensitized drug-containing liposomes were active as anti-angiogenesis agents at very low doses, thus reducing exposure to potentially toxic doses of drug and permitting long term therapy.

### III. EXAMPLES

The following examples further illustrate the invention described herein and are in no way intended to limit the scope of the invention.

### Example 1

In vitro Characterization of Immunoliposomes

A. Liposome Preparation

A mutein of FGF2 (150 amino acids, MW 17,100) was obtained. The mutein contains a single reactive cysteine position. The thiol group of this cysteini was used for coupling with maleimide-polyethylene glycol (2000 Daltons)—disteroyl-glycero-phosphatidyethanolamine, according to known techniques (U.S. Pat. Nos. 6,586,002; 6,326,353), to form an FGF2-PEG-DSPE targeting conjugate.

Liposomes having a coating of polyetheylene glycol chains (PEG, MW 2000 Daltons) were prepared from hydrogenated soy phosphatidyl choline (HSPC), cholesterol, and mPEG-DSPE (molar ratio 50.6144.315.1) were prepared as described in U.S. Pat. No. 5,013,556 to contain doxorubicin (entrapped by ammonium-sulfate promoted remote loading) or as described in U.S. Pat. No. 5,945,122 to contain cisplatin (passively entrapped). 1% of a Texas-Red-DSPE conjugate was included in the liposome formulation to permit tracking of binding and internalization by confocal fluorescence microscopy.

The FGF2-PEG-DSPE targeting conjugate was incorporated into the preformed liposomes via insertion (Uster, P. et al., Febs Lett., 386(2-3):243 (1996)) by incubating FGF2-PEG-DSPE micelles with the preformed liposomes at 60° C. for one hour. Approximately 20 FGF2 targeting ligands per liposome were inserted.

B. In Vitro Studies

Baby hamster kidney (BHK) cells expressing FGF2 receptor were obtained. The cells were incubated with the liposome preparations, washed with saline, and observed via confocal fluorescence microscopy. Cells were incubated (i) at 4° C. for 30 minutes (ii) at 37° C. for 1 hour or 3 hours with immunoliposomes or with identical liposomes lacking the FGF2 targeting ligand (PEGylated liposomes).

Assay with 3-(4,5-dimethylthiazol-2-yl)-2,5-diphenyltetrazolium bromide (MTT assay) was used to determine cell growth inhibition after incubation with the liposome compositions. The results are shown in FIGS. 1A-1F and FIGS. 2A-2B.

### Example 2

In vivo Characterization of Immunoliposomes

Liposomes were prepared as described in Example 1.

Lewis lung carcinoma cells (0.5 million cells) were inocuolated subcutaenously into the flank of B6C3-F1 mice. Paraffin sections of tumor tissues were treated with anti-CD34 receptor antibody to identify endothelial cells and with anti-FGF receptor antibodies to identify cells expressing FGF-receptor. Antibody-bound cells were stained with a secondary horseradish peroxidase-conjugated antibody. The results are shown in FIGS. 3A-3B.

For the anti-tumor therapeutic efficacy study, drug treatment was initiated 15 days after tumor inoculation at doses of 4 mg/kg doxorubicin and 3 mg/kg cisplatin. Immunoliposome treatments were administered once a week for a total of three doses. The results are shown in FIG. 4.

Although the invention has been described with respect to particular embodiments, it will be apparent to those skilled in the art that various changes and modifications can be made without departing from the invention.

It is claimed:

1. A method of treating a disease or disorder characterized by angiogenic tissue growth, comprising
providing an immunoliposome composition comprised of (i) vesicle-forming lipids including between 1-20 mole percent of a vesicle-forming lipid derivatized with a hydrophilic polymer, (ii) a targeting ligand having biological activity to promote angiogenesis, and (iii) a drug entrapped in said liposomes; and
administering said immunoliposome composition to a subject.

2. The method of claim 1, wherein said targeting ligand is a growth factor.

3. The method of claim 2, wherein said growth factor is selected from fibroblast growth factor (FGF), epidermal growth factor (EGF), and vascular endothelial cell growth factor (VEGF).

4. The method of claim 1, wherein said drug is a chemotherapeutic agent.

5. The method of claim 4, wherein said agent is a platinum compound.

6. The method of claim 5, wherein said platinum compound is cisplatin.

7. The method of claim 1, wherein said drug is an angiogenesis inhibitor.

8. The method of claim 7, wherein said angiogenesis inhibitor is a compound selected from the group consisting of FGFR kinase inhibitors, EGFR kinase inhibitors, VEGFR kinase inhibitors, and matrix metalloproteinase inhibitors.

9. The method of claim 1, wherein said polymer is polyethylene glycol.

10. The method of claim 1, wherein said polymer is polyethylene glycol, said ligand is basic fibroblast growth factor, and said drug is a chemotherapeutic agent.

11. The method of claim 10, wherein said chemotherapeutic agent is selected from the group consisting of platinum compounds and vinca alkaloids.

12. The method of claim 1, wherein said polymer is polyethylene glycol, said ligand is basic fibroblast growth factor, and said drug is an angiogenesis inhibitor.
